(19) United States
(12) Patent Application Publication
(10) Pub. No.: US 2008/0081743 A1
(43) Pub. Date: Apr. 3, 2008

(54) Cushion adjusting assembly of an exercise machine

(76) Inventor: Chao-Chuan Chen, Taichung City (TW)

Correspondence Address: Chao-Chuan Chen, 235 Chung-Ho, P.O. Box 8-24, Taipei, omitted

(21) Appl. No.: 11/523,818

(22) Filed: Sep. 19, 2006

(51) Int. Cl. A61F 2/80; A63B 22/06

(52) U.S. Cl. 482/57; 623/38

(57) ABSTRACT

A cushion adjusting assembly of an exercise machine comprises a post; a top end of the post having a combining portion; an adjusting rod and an ejecting unit being pivotally installed in the combining portion; the adjusting rod having at least one inclined surface; and the ejecting unit having at least one inclined surface corresponding to the inclined surface of the adjusting rod; and an adjusting portion having two clamping plates and a stud; a clamping section being formed between the two clamping plates; the stud passing through the two clamping plates so that the two clamping plates are positioned on the combining portion; and the stud passing through the two clamping plates so that the adjusting portion is positioned on the combining portion; the stud passing through the combining portion so as to position the ejecting unit and the adjusting rod.

### FIELD OF THE INVENTION

The present invention relates to exercise machines, and particularly to a cushion adjusting assembly of an exercise machine which is easily adjusted, has a simple structure, can be adjusted easily and conveniently.

### BACKGROUND OF THE INVENTION

In some prior art exercise machine, a post is vertically extended from a body of the machine. A groove seat horizontally extends from the post for installing a cushion by screwing units. The cushion is adjustable along the groove seat. Then the cushion is positioned by screws. Thus the cushion can match the different users.

In above mentioned prior art, the adjusting assembly has a long sliding groove and a groove seat. The manufacturing process is complicated and time consumed. Furthermore the groove seat is made of metal which is expensive.

Moreover, the prior art adjust assembly, the adjust is power consumed. The screw means has short arm of force and thus it is difficult to adjust it by hands. Tools are necessary. Thus it is inconvenient.

### SUMMARY OF THE INVENTION

Accordingly, the primary object of the present invention is to provide a cushion adjusting assembly of an exercise machine which is easily adjusted, has a simple structure, can be adjusted easily and conveniently.

To achieve above objects, the present invention provides a cushion adjusting assembly of an exercise machine which comprises a post; a top end of the post having a combining portion; an adjusting rod and an ejecting unit being pivotally installed in the combining portion; the adjusting rod having at least one inclined surface; and the ejecting unit having at least one inclined surface corresponding to the inclined surface of the adjusting rod; and an adjusting portion having two clamping plates and a stud; a clamping section being formed between the two clamping plates; the stud passing through the two clamping plates so that the two clamping plates are positioned on the combining portion; and the stud passing through the two clamping plates so that the adjusting portion is positioned on the combining portion; the stud passing through the combining portion so as to position the ejecting unit and the adjusting rod.

The various objects and advantages of the present invention will be more readily understood from the following detailed description when read in conjunction with the appended drawing.

### BRIEF DESCRIPTION OF THE DRAWINGS

FIG. 1 is an exploded perspective view of the cushion adjusting assembly of an exercise machine of the present invention.

FIG. 2 is a schematic perspective view of the cushion adjusting assembly of an exercise machine of the present invention.

FIG. 3 is a cross sectional view of the cushion adjusting assembly of an exercise machine of the present invention.

FIG. 4 is a schematic cross sectional view of the cushion adjusting assembly of an exercise machine of the present invention.

FIGS. 5 and 6 are schematic view showing the adjustment of the cushion adjusting assembly of an exercise machine of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

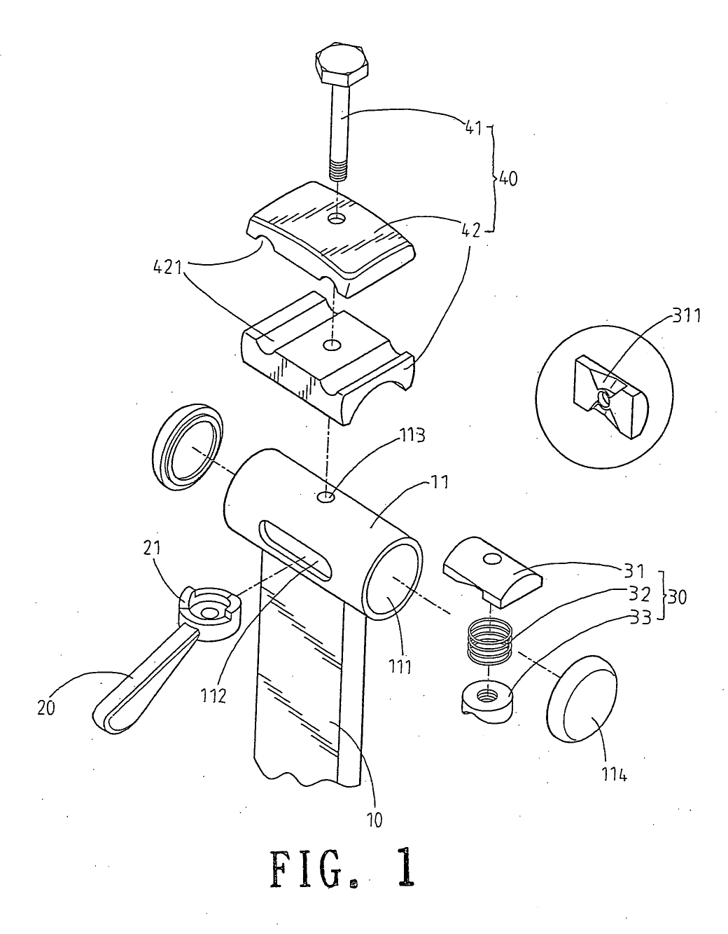

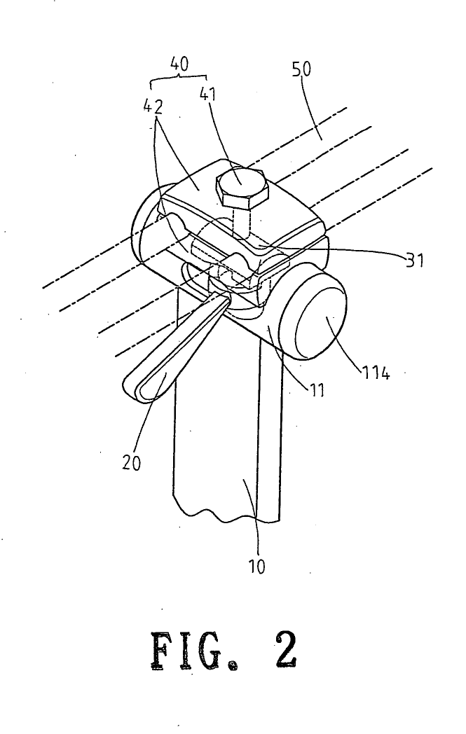

A cushion adjusting assembly of an exercise machine of the present invention is illustrated in FIGS. 1 and 2. The present invention has the following elements.

A post 10 is installed on an exercise machine. A top end of the post 10 has a combining portion 11. An inner side of the combining portion 11 has a receiving space 111 for pivotally installing an adjusting rod 20 and an elastic unit 32. A lateral side of the combining portion 11 is formed with an adjusting hole 112 which is communicated to the receiving space 111. The adjusting rod 20 protrudes from the adjusting hole 112. A top of the combining portion 11 has a through hole 113 for receiving a stud 41 of the adjusting portion 40. Two ends of the receiving space 111 of the combining portion 11 are sealed by two outer covers 114.

The adjusting rod 20 is pivotally installed in the receiving space 111. One end of the adjusting rod 20 protrudes from the combining portion 11. Another end of the adjusting rod 20 is a ring 21 with inclined surfaces at an upper side thereof.

The ejecting unit 30 has a resisting block 31, an elastic unit 32 and a nut 33. Thus the user can rotate the adjusting rod 20 to push an ejecting unit 30. The resisting block 31 has inner concave inclined surfaces corresponding to the inclined surfaces of the adjusting rod 20. A top end of the resisting block 31 adheres upon an inner surface of the receiving space 111. The elastic unit 32 is installed between the resisting block 31 and the nut 32 for providing an elastic resilient function to the elastic unit 32.

The adjusting portion 40 includes the stud 41, and two clamping plates 42. The stud 41 is corresponding to the nut 33 of the elastic unit 32. The stud 41 passes through the two clamping plates 42 and then inserts into the receiving space 111 through the through hole 113 of the combining portion 11 and then passes through the resisting block 31, the adjusting rod 20, and the elastic unit 32 so as to be positioned in the nut 33. Thereby the adjusting rod 20 and the elastic unit 32 are pivotally installed in the receiving space 111. A clamping section 421 is formed between the two clamping plates 42 for positioning a positioning rod 50 of the cushion. When the two clamping plates 42 are tightly engaged to one another, the positioning rod 50 is retained. When the clamping plates 42 are released, the positioning rod 50 is movable so as to adjust the position of the cushion.

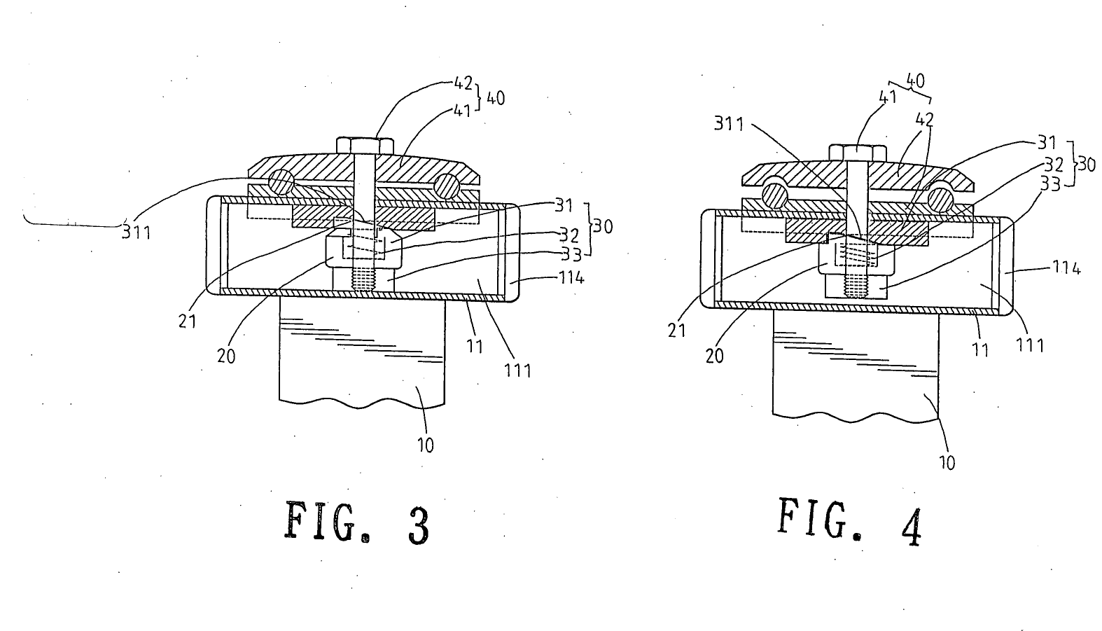

In use, referring to FIGS. 3 to 6, when the adjusting rod 20 rotates so that the inclined surfaces 21 of the adjusting rod 20 is at a highest position to resist against the shallowest areas of the inclined surfaces 311 of the resisting block 31, the adjusting rod 20 and the resisting block 31 will expand slightly so that the nut 33 of the ejecting unit 30 will move downwards. Then the stud 31 locked to the nut 33 will move downwards also. By the stud 41 the two clamping plates 42 will clamp tightly so that the positioning rod 50 in the clamping section 421 between two clamping plates 42 are clamped therein and then are fixed therein.

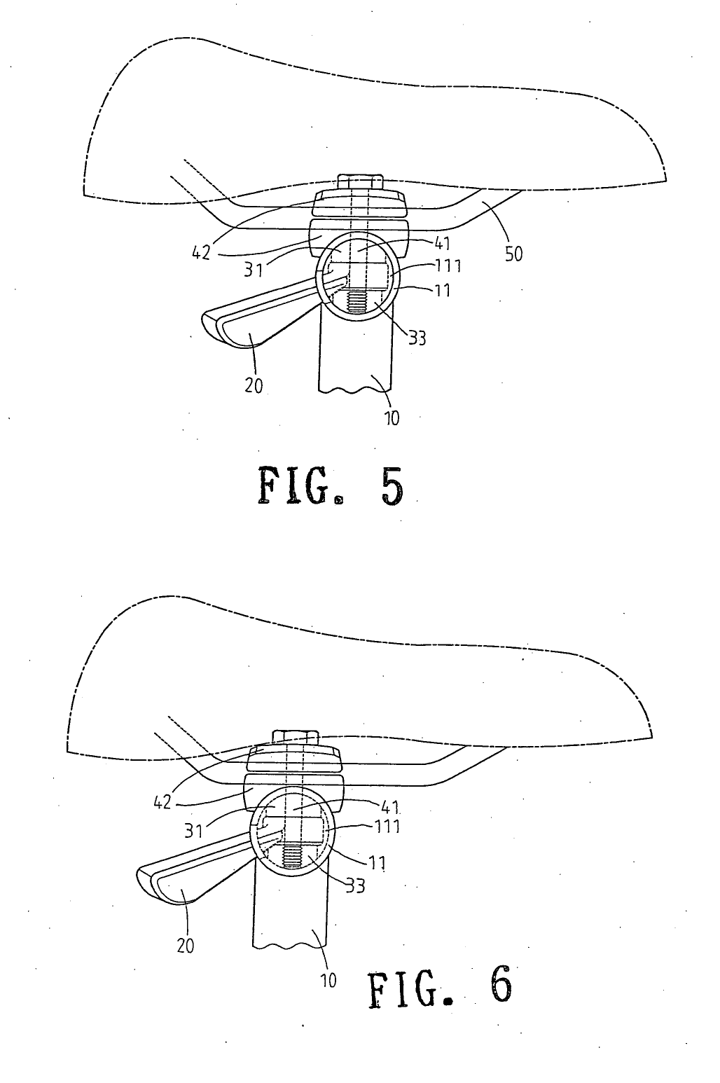

Then, when the user needs to adjust the position of the cushion, the adjusting rod 20 moves to another side so that the lowest portion of the inclined surfaces 21 of the adjusting rod 20 resists against the deepest position of the inclined surfaces 311 of the resisting block 31. The adjusting rod 20 and resisting block 31 will reduce so as to provide a space for moving the nut 33 upwards. Then the stud 41 has a space for moving upwards. Then the two clamping plates 42 release, the adjusting rod 20 in the clamping section 421 between the two clamping plates 42 is not limited so that as referring to FIGS. 5 and 6, the positioning rod 50 moves forwards and backwards along the clamping section 421. After the user adjusts the adjusting rod 20, the adjusting rod 20 moves reversely so that the two clamping plates 42 of the adjusting portion 40 are tightly clamped by the stud 41 by using the adjusting rod 20 and the ejecting unit 30. Thus the cushion is fixed to a desired position.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A cushion adjusting assembly of an exercise machine, comprising:
a post; a top end of the post having a combining portion; an adjusting rod and an ejecting unit being pivotally installed in the combining portion; the adjusting rod having at least one inclined surface; and the ejecting unit having at least one inclined surface corresponding to the inclined surface of the adjusting rod; and
an adjusting portion having two clamping plates and a stud; a clamping section being formed between the two clamping plates; the stud passing through the two clamping plates so that the two clamping plates are positioned on the combining portion; and the stud passing through the two clamping plates so that the adjusting portion is positioned on the combining portion; the stud passing through the combining portion to position the ejecting unit and the adjusting rod.

2. The cushion adjusting assembly of an exercise machine as claimed in claim 1, wherein the ejecting unit has a resisting block, an elastic unit and a nut; the resisting block has at least one inclined surface corresponding to the inclined surface of the adjusting rod; the elastic unit is installed between the nut and the resisting block; and the nut can lock the stud.

3. The cushion adjusting assembly of an exercise machine as claimed in claim 1, wherein a receiving space is formed in the combining portion for installing the adjusting rod and the ejecting unit and the combining portion has an adjusting hole; one end of the adjusting rod protrudes out of the adjusting rod.

4. The cushion adjusting assembly of an exercise machine as claimed in claim 1, wherein the post is installed on an exercise machine; and a cushion is clamped in the clamping section of the clamping plates.

5. The cushion adjusting assembly of an exercise machine as claimed in claim 4, wherein a bottom of the cushion has a positioning rod which is installed in the clamping section.
